(19) United States
(12) Patent Application Publication
(10) Pub. No.: US 2023/0287524 A1
(43) Pub. Date: Sep. 14, 2023

(54) COMPOSITION, KIT AND METHOD FOR DETECTING AND CLASSIFYING PATHOGENS CAUSING RESPIRATORY TRACT INFECTION, AND APPLICATION

(71) Applicant: SANSURE BIOTECH INC., Changsha (CN)

(72) Inventors: Lizhong DAI, Changsha (CN); Xiaoliang CHEN, Changsha (CN); Xiaomei REN, Changsha (CN); Jiangyan XIANG, Changsha (CN); Wang WAN, Changsha (CN); Shiliu LIAO, Changsha (CN); Zhongping DENG, Changsha (CN)

(21) Appl. No.: 18/045,616

(22) Filed: Oct. 11, 2022

Related U.S. Application Data
(63) Continuation of application No. PCT/CN2020/121011, filed on Oct. 15, 2020.

(30) Foreign Application Priority Data
Apr. 16, 2020 (CN) 202010302240.2

(51) Int. Cl. C12Q 1/70; C12Q 1/6851

(52) U.S. Cl. CPC C12Q 1/701; C12Q 1/6851; C12Q 2600/166

(57) ABSTRACT

Provided is a composition for detecting novel coronavirus 2019-nCoV, influenza A virus, influenza B virus, respiratory adenovirus, respiratory syncytial virus, and Mycoplasma pneumoniae. Also provided are a kit containing the composition, a use of the composition, and a method for detecting and typing pathogens that cause a respiratory infection.

### CROSS-REFERENCE TO RELATED APPLICATIONS

This application is a continuation of International Application No. PCT/CN2020/121011, filed on Oct. 15, 2020, which claims priority to Chinese Patent Application No. 202010302240.2, filed on Apr. 16, 2020. The disclosures of the aforementioned applications are hereby incorporated by reference in their entireties.

### REFERENCE TO AN ELECTRONIC SEQUENCE LISTING

The contents of the electronic sequence listing (CU710SequenceListing.xml; Size: 31,273 bytes; and Date of Creation: Oct. 11, 2022) is herein incorporated by reference in its entirety.

### TECHNICAL FIELD

The present invention pertains to the field of molecular biological detection, specifically, to the field of detection of pathogens that cause respiratory tract infections. More specifically, the present invention is capable of simultaneous detection and typing of novel coronavirus 2019-nCoV, influenza A virus, influenza B virus, a respiratory adenovirus, a respiratory syncytial virus, and Mycoplasma pneumoniae.

### BACKGROUND

Infectious respiratory system diseases are mostly common and frequently-occurring diseases in clinical practice. These diseases have similar clinical symptoms and epidemiological characteristics, and it is difficult to identify and determine the type of pathogens infected from clinical symptoms and by routine laboratory tests. Respiratory pathogens can be transmitted through the air. Pathogens that cause acute respiratory diseases have the characteristics of strong infectivity, rapid transmission, short incubation periods, and acute onset, and may cause a wide range of acute upper and lower respiratory tract diseases and seriously endanger human health. Common respiratory tract pathogens include the following pathogens:

2019 novel coronavirus (2019-nCoV) was discovered in December 2019, and was subsequently named SARS-CoV-2 by the International Committee on Taxonomy of Viruses on February 11th. Coronaviruses are a large family of viruses. Six species were previously known to infect humans, such as those that can cause colds as well as Middle East Respiratory Syndrome (MERS) and Severe Acute Respiratory Syndrome (SARS). The novel coronavirus 2019-nCoV is a new strain of coronavirus that has never been found in humans before. Taxonomically, the virus is a (3-type coronavirus in the genus Coronavirus of the family Coronaviridae, and has an envelope and spikes, and is a linear positive-sense single-stranded RNA ((+)ssRNA) virus according to the genomic cytological characteristic. Since the discovery of the novel coronavirus 2019-nCoV in December 2019, the virus has almost exploded all over the world in just over three months, infecting more than one million people globally and killing more than 50,000 people, raising a great attention in the international community.

Influenza virus, briefly referred to as flu virus, is an RNA virus that causes influenza in humans and animals. Human influenza viruses are divided into three types: types A, B, and C, which are pathogens of influenza (flu). AI, short for avian influenza, is an infectious disease caused by a certain subtype of influenza A virus (also called avian influenza virus). Taxonomically, influenza viruses belong to the genus Influenzavirus of the family Orthomyxoviridae, and will cause acute upper respiratory infections, and spread rapidly through the air, and often cause periodic pandemics around the world, such as the “Spanish Influenza” that killed more than 20 million people worldwide in 1918-1919, the “Asian Influenza” that occurred in 1957, the “Hong Kong Influenza” that occurred in 1968, the “Russian Influenza” occurred in 1977, and H7N9 bird flu occurred in 2013.

Respiratory Syncytial Virus (RSV) is an RNA virus belonging to the genus Pneumonia of the family Paramyxoviridae. Clinical studies have shown that the RSV is the most common pathogen that causes viral pneumonia, and is more common in infants under three years of age, showing the main symptoms of high fever, rhinitis, pharyngitis, and laryngitis, which later develop into bronchiolitis and pneumonia. A small number of sick children may be complicated by otitis media, pleurisy, and myocarditis. For infected adults and older children, the main manifestation is upper respiratory infections.

Adenoviruses (HAdv) belong to the family Adenoviridae. According to different immunological, biological, and biochemical characteristics, they are divided into seven subspecies from A to G, a total of 52 serotypes. Different serotypes have different organ affinities and cause corresponding clinical manifestations, and may infect the respiratory tract, gastrointestinal tract, urethra, bladder, eyes, and liver.

Mycoplasma pneumoniae (MP) is a common pathogen of community-acquired pneumonia. The infection with 1VIP may cause atypical pneumonias. The pathological changes are mainly interstitial pneumonia, sometimes complicated by bronchial pneumonia. 1VIP is mainly transmitted by droplets, with an incubation period of two to three weeks, and the incidence is highest in adolescents. The clinical symptoms are mild, and most patients only have general respiratory tract symptoms such as headache, sore throat, fever, and cough; a few will have persistent high fever and severe cough, show rapid disease progression, and develop into respiratory failure, multiple organ dysfunction, and other critical illnesses in a short period of time.

Infectious respiratory tract diseases have similar clinical symptoms and epidemiological characteristics, and early diagnosis, early treatment, and early isolation are the fundamentals for treatment and prevention. However, it is difficult to identify and determine the type of pathogens infected from clinical symptoms and by routine laboratory tests, and the culture conditions of pathogens are critical, resulting in a low positive rate of culture, and some pathogens even cannot be cultured under current conditions, which pose great difficulty for patients with respiratory infections and clinicians.

Therefore, there is an urgent need in the art for a simple, rapid, and objective method for detecting a respiratory pathogen, so as to achieve pathogen determination, early diagnosis, and rational guidance of clinical medication.

### SUMMARY

In view of this, provided in the present application is a composition capable of detecting and typing pathogens that cause a respiratory infection. The composition includes:
a first nucleic acid composition:
an influenza A virus forward primer as shown in SEQ ID NO: 1, an influenza A virus reverse primer as shown in SEQ ID NO: 2, and an influenza A virus probe as shown in SEQ ID NO: 3;
an influenza B virus forward primer as shown in SEQ ID NO: 4, an influenza B virus reverse primer as shown in SEQ ID NO: 5, and an influenza B virus probe as shown in SEQ ID NO: 6;
a respiratory syncytial virus forward primer as shown in SEQ ID NO: 7, a respiratory syncytial virus reverse primer as shown in SEQ ID NO: 8, and a respiratory syncytial virus probe as shown in SEQ ID NO: 9; and
a second nucleic acid composition:
a respiratory adenovirus forward primer as shown in SEQ ID NO: 10, a respiratory adenovirus reverse primer as shown in SEQ ID NO: 11, and a respiratory adenovirus probe as shown in SEQ ID NO: 12;
a novel coronavirus 2019-nCoV forward primer as shown in SEQ ID NO: 13, a novel coronavirus 2019-nCoV reverse primer as shown in SEQ ID NO: 14, and a novel coronavirus 2019-nCoV probe as shown in SEQ ID NO: 15;
a Mycoplasma pneumoniae forward primer as shown in SEQ ID NO: 16, a Mycoplasma pneumoniae reverse primer as shown in SEQ ID NO: 17, and a Mycoplasma pneumoniae probe as shown in SEQ ID NO: 18;
wherein fluorescent reporter groups of the first nucleic acid composition are different from each other and do not interfere with each other, and fluorescent reporter groups of the second nucleic acid composition are different from each other and do not interfere with each other.

Herein, the description “different from each other and do not interfere with each other” means that the fluorescent reporter groups used by the respective probes in the nucleic acid composition are different, and will not affect the detection of each other, i.e., different channels may be used for detection. For example, FAM, HEX, ROX, and CY5 may be used. The absorbance values of these groups are not close, and different channels may be selected, and therefore, the groups will not interfere with each other.

Further, the composition includes: an internal standard forward primer, internal standard reverse primer, and internal standard probe for monitoring.

In one specific embodiment, the composition further includes: the internal standard forward primer as shown in SEQ ID NO: 19, the internal standard reverse primer as shown in SEQ ID NO: 20, and the internal standard probe as shown in SEQ ID NO: 21.

In the present invention, the fluorescent reporter group may be selected from FAM, HEX, ROX, VIC, CY5, 5-TAMRA, TET, CY3, and JOE, but is not limited thereto.

In one specific embodiment, a fluorescent reporter group of the influenza A virus probe as shown in SEQ ID NO: 3 is FAM; a fluorescent reporter group of the influenza B virus probe as shown in SEQ ID NO: 6 is HEX; and a fluorescent reporter group of the respiratory syncytial virus probe as shown in SEQ ID NO: 9 is CY5.

In one specific embodiment, a fluorescent reporter group of the respiratory adenovirus probe as shown in SEQ ID NO: 12 is FAM; a fluorescent reporter group of the novel coronavirus 2019-nCoV probe as shown in SEQ ID NO: 15 is HEX; and a fluorescent reporter group of the Mycoplasma pneumoniae probe as shown in SEQ ID NO: 18 is CY5.

In one specific embodiment, a fluorescent reporter group of the internal standard probe as shown in SEQ ID NO: 21 is ROX.

The primer and probe of the internal standard detect the gene sequence of a human housekeeping gene GAPDH. The GAPDH gene sequence (SEQ ID NO: 22) is:

TCTCCTCTGACTTCAACAGCGACACCCACTCCTCCACCTTTGACGCTGGG
GCTGGCATTGCCCTCAACGACCACTTTGTCAAGCTCATTTCCTGGTATGA
CAACGAATTTGGCTACAGCAACAGGGTGGTGG

Further, an amount of the detection primer in the composition is 25 nM-500 nM; an amount of the detection probe in the composition is 20 nM-500 nM; an amount of the internal standard primer in the composition is 12.5 nM-250 nM; and an amount of the internal standard probe in the composition is 10 nM-250 nM.

In the present invention, the terms “detection primer” and “detection probe” refer to a primer and a probe used for amplifying and detecting a pathogen.

In the present invention, the terms “internal standard primer” and “internal standard probe” refer to a primer and a probe used for amplifying and detecting the internal standard.

In one specific embodiment, two nucleic acid compositions of the composition of the present invention are each present in separate packages.

Further, the components of each nucleic acid composition of the composition of the present invention are present in a mixed form.

In a second aspect, provided in the present application is a use of the above composition of the present invention in the preparation of a kit for detecting and typing pathogens that cause a respiratory infection.

The pathogens that cause the respiratory infection include: a novel coronavirus 2019-nCoV, an influenza A virus, an influenza B virus, a respiratory adenovirus, a respiratory syncytial virus, and Mycoplasma pneumoniae.

In a third aspect, provided in the present application is a kit for detecting and typing pathogens that cause a respiratory infection, the kit including the above composition of the present invention.

Further, the kit further includes at least one of a nucleic acid release reagent, a dNTP, a reverse transcriptase, a DNA polymerase, a PCR buffer, and Mg²⁺.

A common PCR buffer is composed of a buffer system such as Tris-HCl, MgCl₂, KCl, and Triton X-100. Generally, the total volume in a single PCR reaction tube is 20 μL-200 μL.

Further, the amount of the detection primer in the composition is 25 nM-500 nM; the amount of the detection probe in the composition is 20 nM-500 nM; the amount of the internal standard primer in the composition is 12.5 nM-250 nM; the amount of the internal standard probe in the composition is 10 nM-250 nM; and an amount of the dNTP is 0.2 mM-0.3 mM.

Further, a concentration of the reverse transcriptase is 5 U/μL to 15 U/μL, and for example, the reverse transcriptase may be a murine leukemia reverse transcriptase (MMLV) or a Tth enzyme. A concentration of the DNA polymerase is 5 U/μL to 15 U/μL, and for example, the DNA polymerase may be a Taq enzyme.

In one specific embodiment, the PCR system of the composition of the present invention is as follows:

| Component | Volume/concentration in each reaction | |
|---|---|---|
| 5 x PCR buffer* | 10 | μL |
| dNTPs (100 mM) | 1.5 | μL |
| Tth enzyme (5 U/μL) | 1.0 | μL |
| H-Taq enzyme (5 U/μL) | 1.0 | μL |
| Mn(OAc)₂ (150 mM) | 1.0 | μL |
| Detection primer | 0.5 | μM |
| Detection probe | 0.25 | μM |
| Internal standard primer | 0.25 | μM |
| Internal standard probe | 0.1 | μM |

| | |
|---|---|
| Sterilized purified water | Make up to 45 μL |

Further, the kit contains a positive control and a negative control, and the negative and positive controls and the testing sample need to be processed simultaneously. The positive control is formed by mixing an artificially synthesized lentiviral particle containing specific nucleic acid sequences of a 2019 novel coronavirus, an influenza A virus, an influenza B virus, and a respiratory syncytial virus with an artificially synthesized plasmid containing specific nucleic acid sequences of an adenovirus and Mycoplasma pneumoniae, to simulate an actual clinical sample. The negative control consists of an artificially synthesized lentiviral particle containing a target fragment of a GAPDH housekeeping gene (SEQ ID NO: 22), to completely simulate a throat swab sample of a normal person.

In a fourth aspect, a method for detecting and typing pathogens that cause a respiratory infection is provided, the method including the steps of:
1) releasing a nucleic acid of a testing sample;
2) performing, by using the above composition of the present invention or the above kit of the present invention, a fluorescent quantitative PCR on the nucleic acid obtained in step 1); and
3) obtaining and analyzing results.

In the present invention, the testing sample may be a throat swab, sputum, a bronchoalveolar lavage fluid, blood, etc., but is not limited thereto.

Further, reaction conditions of the fluorescent quantitative PCR are as follows:

pre-denaturation and enzyme activation at a temperature of 95° C. for 1-10 minutes for 1 cycle; reverse transcription at a temperature of 60° C. for 25-35 minutes for 1 cycle; cDNA pre-denaturation at a temperature of 95° C. for 1-10 minutes for 1 cycle; denaturation at a temperature of 95° C. for 10-20 seconds; annealing at a temperature of 60° C. for 20-40 seconds for 40-50 cycles.

In one specific embodiment, a method for detecting and typing pathogens that cause a respiratory infection for a non-diagnostic purpose is provided, the method including the steps of:
1) releasing a nucleic acid of a testing sample;
2) performing, by using the above composition of the present invention or the above kit of the present invention, a fluorescent quantitative PCR on the nucleic acid obtained in step 1); and
3) obtaining and analyzing the results.

Using the composition of the present invention can simultaneously detect and type the six pathogens that cause respiratory infections, so as to identify some patients having fever caused by common seasonal influenza, reduce the waste of medical resources, and reduce the psychological burden on patients and society.

Furthermore, the present invention can quickly diagnose the novel coronavirus 2019-nCoV, provide molecular evidence for early diagnosis, early treatment, and early isolation of the novel coronavirus, and allow adequate preparations for prevention and control of the disease, making it possible to control the source of infection of the highly contagious and hazardous novel coronavirus 2019-nCoV in a timely manner and stop virus pandemics and outbreaks.

The composition of the present invention in combination with a fluorescent probe method enables the use of two tubes in one test simultaneously, achieving low costs and high throughput. The present invention enables information of four targets to be given by one tube in a single test, and the operations are simple and convenient, and a result reading process can be completed according to a CT value. The whole detection process is carried out under single-tube closed conditions, avoiding false positives and environmental contamination caused by crossover between samples.

### BRIEF DESCRIPTION OF DRAWINGS

FIG. 1 is a detection diagram of the composition of the present invention for detecting a novel coronavirus in 400 copies/mL;

FIG. 2 is a sensitivity detection diagram of the composition of the present invention for detecting an influenza A virus in 6.5 TCID50/mL;

FIG. 3 is a sensitivity detection diagram of the composition of the present invention for detecting an influenza B virus in 3.0 TCID50/mL;

FIG. 4 is a detection diagram of the composition of the present invention for detecting a respiratory syncytial virus in 500 copies/mL;

FIG. 5 is a detection diagram of the composition of the present invention for detecting a respiratory adenovirus in 400 copies/mL;

FIG. 6 is a detection diagram of the composition of the present invention for detecting Mycoplasma pneumoniae in 400 copies/mL;

FIG. 7 is a specificity detection diagram of the composition of the present invention;

FIG. 8 shows an effect of an interfering substance on detection of a novel coronavirus (including a control curve);

FIG. 9 shows an effect of an interfering substance on detection of an influenza A virus (including a control curve);

FIG. 10 shows an effect of an interfering substance on detection of an influenza B virus (including a control curve);

FIG. 11 shows an effect of an interfering substance on detection of a respiratory syncytial virus (including a control curve);

FIG. 12 shows an effect of an interfering substance on detection of an adenovirus (including a control curve);

FIG. 13 shows an effect of an interfering substance on detection of Mycoplasma pneumoniae (including a control curve);

FIG. 14 is a detection result diagram, in which A shows the result of the composition of the present invention, and B shows the result when a rhinovirus is used instead of Mycoplasma pneumoniae in the present invention;

FIG. 15 is a detection result diagram, in which A shows the result of the composition of the present invention, and B shows the result when a metapneumovirus is used instead of Mycoplasma pneumoniae in the present invention;

FIG. 16 is a detection diagram of the composition of the present invention for detecting six influenza A viruses;

FIG. 17 is a detection diagram of the composition of the comparative example of the present invention for detecting six influenza A viruses;

FIG. 18 is a detection diagram of the composition of the present invention for detecting a novel coronavirus 2019-nCoV in three gradients (2000 copies/mL, 500 copies/mL, and 200 copies/mL); and

FIG. 19 is a detection diagram of a comparative example composition of the present invention for detecting a novel coronavirus 2019-nCoV in three gradients (2000 copies/mL, 500 copies/mL, and 200 copies/mL).

### DETAILED DESCRIPTION

The present invention will be described in detail below with reference to specific embodiments and examples, and the advantages and various effects of the present invention will be more clearly presented therefrom. It should be understood by those skilled in the art that these specific embodiments and examples are intended to illustrate the present invention, rather than to limit the present invention.

### Example 1. Primers and Probes Used in the Present Invention

The primers and probes used in the present invention are shown in Table 1 below:

TABLE 1

| Name | Base sequence 5′-3′ |
|---|---|
| Influenza A virus forward primer | CAGAAGATTTGTCCTTCCAG |
| SEQ ID NO: 1 | |
| Influenza A virus reverse primer | GTCATACTCCTCTGCATTGTCTC |
| SEQ ID NO: 2 | |
| Influenza A virus probe | TCTTCGAGCTCTCGGACGAAAAGGCAA |
| SEQ ID NO: 3 | |
| Influenza B virus forward primer | GCCATCGGATCCTCAATTC |
| SEQ ID NO: 4 | |
| Influenza B virus reverse primer | TTCTTCCGTGACCAGTCTAATT |
| SEQ ID NO: 5 | |
| Influenza B virus probe | AAGGACATCCAAAGCCAATTCGAGCAG |
| SEQ ID NO: 6 | |
| Respiratory syncytial virus forward primer | CACCCTGACAAAATCAACAATATA |
| SEQ ID NO: 7 | |
| Respiratory syncytial virus reverse primer | CATAGGCACCCATATTGTTAGT |
| SEQ ID NO: 8 | |
| Respiratory syncytial virus probe | AGACAAGATGGGGCAAATATGGAAAC |
| SEQ ID NO: 9 | |
| Respiratory adenovirus forward primer | GCAGTGGTCTTACATGCACATC |
| SEQ ID NO: 10 | |
| Respiratory adenovirus reverse primer | TATCCTCACGGTCCACAGGG |
| SEQ ID NO: 11 | |
| Respiratory adenovirus probe | CCAGGACGCCTCGGAGTACCTGAGC |
| SEQ ID NO: 12 | |
| Novel coronavirus 2019-nCoV forward primer | AGCAATCTTTAATCAGTGTGTAACAT |
| SEQ ID NO: 13 | |
| Novel coronavirus 2019-nCoV reverse primer | AAATCACATGGGGATAGCACTACT |
| SEQ ID NO: 14 | |
| Novel coronavirus 2019-nCoV probe | AAAGAGCCACCACATTTTCACCGAGG |
| SEQ ID NO: 15 | |
| Mycoplasma pneumoniae forward primer | CTTTGGTTCGCATGAATCAA |
| SEQ ID NO: 16 | |
| Mycoplasma pneumoniae reverse primer | TTCCCTACTGCTGCCTCC |
| SEQ ID NO: 17 | |
| Mycoplasma pneumoniae probe | TCGTTATTTGATGAGGGTGCGCCATATC |
| SEQ ID NO: 18 | |
| Internal standard forward primer | CTCCTCTGACTTCAACAGCGA |
| SEQ ID NO: 19 | |
| Internal standard reverse primer | CCACCACCCTGTTGCTGT |
| SEQ ID NO: 20 | |
| Internal standard probe | TTGACGCTGGGGCTGGCATT |
| SEQ ID NO: 21 | |

Among them, fluorescent reporter groups of the influenza A virus probe and the respiratory adenovirus probe were FAM; fluorescent reporter groups of the novel coronavirus 2019-nCoV probe and the influenza B virus probe were HEX; fluorescent reporter groups of the respiratory syncytial virus and the Mycoplasma pneumoniae probe were CY5; and a fluorescent reporter group of the internal standard was ROX, and the 3′-end of the probe further had a BHQ1 or BHQ2 quencher group.

### Example 2. Method for Detecting and Typing Pathogens that Cause a Respiratory Infection

A testing sample of the present invention was a throat swab, sputum, a bronchoalveolar lavage fluid, or blood. A magnetic bead method was used to extract a viral nucleic acid, and the following operations were performed in a sample processing room:

2.1 An appropriate number of 1.5 mL sterilized centrifuge tubes were taken, and labeled as a negative control, a positive control, and a testing sample, respectively. 300 μL of an RNA extraction solution 1 was added to each tube.

2.2 200 μL of the testing sample or the negative control or the positive control was added to each tube. The tube was covered with a cap, and shaken for 10 seconds for through mixing, and subjected to instant centrifugation.

2.3 100 μL of an RNA extraction solution 2-mix was added to each tube (sucked up after through mixing), and the tube was shaken for 10 seconds for through mixing, and left to stand for 10 minutes at room temperature.

2.4 After instant centrifugation, the centrifuge tubes were placed on a separator, and the solution was slowly sucked out after 3 minutes (be careful not to touch a brown substance adhered to the tube wall).

2.5 600 μL of an RNA extraction solution 3 and 200 μL of an RNA extraction solution 4 were added to each tube, and the tube was shaken for 5 seconds for through mixing and subjected to instant centrifugation, and then the centrifuge tube was placed on the separator again.

2.6 After about 3 minutes, the supernatant separated into two layers. A pipette tip was inserted into the bottom of the centrifuge tube, the liquid was slowly sucked up from the bottom completely and discarded. The tube was left to stand for 1 minute and then the residual liquid at the bottom of the tube was completely sucked up and discarded.

2.7 50 μL of a TE buffer (pH 8.0) was added to each tube to perform elution, and then all the eluted brown mixture was transferred as a testing sample to a 0.2 mL PCR reaction tube, and the tube was covered with a cap and transferred to an amplification test zone.

The real-time fluorescent PCR reaction system was configured as follows:

| Component | Volume/concentration in each reaction | |
|---|---|---|
| 5 x PCR buffer | 10 | μL |
| dNTPs (100 mM) | 1.5 | μL |
| Tth enzyme (5 U/μL) | 1.0 | μL |
| H-Taq enzyme (5 U/μL) | 1.0 | μL |
| Mn(OAc)₂ (150 mM) | 1.0 | μL |
| Detection primer | 0.5 | μM |
| Detection probe | 0.25 | μM |
| Internal standard primer | 0.25 | μM |
| Internal standard probe | 0.1 | μM |

| | |
|---|---|
| Sterilized purified water | Make up to 45 μL |

The PCR amplification program was set up as follows:

| | | | | | Number of |
|---|---|---|---|---|---|
| Step | | Temperature | Time | | cycles |
| 1 | Pre-denaturation and | 95° C. | 1 | minute | 1 |
| | enzyme activation | | | | |
| 2 | Reverse transcription | 60° C. | 30 | minutes | 1 |
| 3 | cDNA pre-denaturation | 95° C. | 1 | minute | 1 |
| 4 | Denaturation | 95° C. | 15 | seconds | 45 |
| | Annealing, extension, | 60° C. | 30 | seconds | |
| | and fluorescence | | | | |
| | collection | | | | |
| 5 | Instrument cooling | 25° C. | 10 | seconds | 1 |

Result analysis:
1) Target detection signals were FAM, HEX (or VIC), and CYS, and an internal reference detection signal was ROX.
2)Baseline setting: The baseline was generally set to 3-15 cycles, which specifically could be adjusted according to actual situations. The adjustment principle was to select a region where a fluorescence signal is relatively stable before exponential amplification, a starting point (Start) avoiding signal fluctuation in an initial stage of fluorescence collection, and an ending point (End) being less by 1-2 cycles than the Ct of a sample showing the earliest exponential amplification. Threshold setting: The setting principle was to make a threshold line just exceed the highest point of a normal negative control.
3) It was first analyzed whether an amplification curve is detected for the internal standard in the ROX channel and Ct<39, and if so, it indicated that the current test was effective, and subsequent analysis continued to be carried out:
A) if a typical S-shaped amplification curve was detected in the FAM channel and Ct<39, it indicated that the influenza A virus and respiratory adenovirus detection results were positive;
B) if a typical S-type amplification curve was detected in the HEX channel and Ct<39, it indicated that the novel coronavirus 2019-nCoV and influenza B virus detection results were positive; and
C) if a typical S-type amplification curve was detected in the CY5 channel and Ct<39, it indicated that the respiratory syncytial virus and Mycoplasma pneumoniae detection results were positive;
4) If a Ct for the internal standard was not detected in the ROX channel or Ct>39, it indicated that the concentration of the testing sample was excessively low or there was an interfering substance that inhibited the reaction, and the experiment needed to be re-prepared.

### Example 3. Detection Results of Clinical Samples Tested by the Composition of the Present Invention

The composition in Table 1 of the present invention was used to test 411 specimens according to the method described in Example 2, the results were one 2019 novel coronavirus cell culture solution, 25 positive cases of influenza A virus, 68 positive cases of influenza B virus, 77 positive cases of respiratory adenovirus, 18 positive cases of respiratory syncytial virus, and 27 positive cases of Mycoplasma pneumoniae. The overall results were consistent with those identified by the Center for Disease Control and Prevention. Additionally, the composition of the present invention was compared with a control method (a method using a commercially available kit as a control kit). While the control method had false negatives or false positives, the detection effects of the composition of the present invention were more accurate. The results are shown in Tables 2-7 below.

TABLE 2
Influenza A virus (flu A) negative and positive statistical table

Control method

| | | | |
|---|---|---|---|
| Experiment | Positive | Negative | Total |

| | | | | |
|---|---|---|---|---|
| Kit to be assessed | Positive | 25 | 0 | 25 |
| | Negative | 0 | 385 | 385 |
| Total | | 25 | 385 | 410 |

TABLE 3
Influenza B virus (flu B) negative and positive statistical table

Control method

| | | | |
|---|---|---|---|
| Experiment | Positive | Negative | Total |

| | | | | |
|---|---|---|---|---|
| Kit to be assessed | Positive | 67 | 1 | 68 |
| | Negative | 1 | 341 | 342 |
| Total | | 68 | 342 | 410 |

TABLE 4
Respiratory syncytial virus (RSV) negative
and positive statistical table

Control method

| | | | |
|---|---|---|---|
| Experiment | Positive | Negative | Total |

| | | | | |
|---|---|---|---|---|
| Kit to be assessed | Positive | 18 | 2 | 20 |
| | Negative | 0 | 390 | 390 |
| Total | | 18 | 392 | 410 |

TABLE 5
Adenovirus (ADV) negative and positive statistical table

Control method

| | | | |
|---|---|---|---|
| Experiment | Positive | Negative | Total |

| | | | | |
|---|---|---|---|---|
| Kit to be assessed | Positive | 76 | 11 | 87 |
| | Negative | 1 | 322 | 323 |
| Total | | 77 | 333 | 410 |

TABLE 6
Mycoplasma pneumoniae (MP) negative
and positive statistical table

Control method

| | | | |
|---|---|---|---|
| Experiment | Positive | Negative | Total |

| | | | | |
|---|---|---|---|---|
| Kit to be assessed | Positive | 27 | 2 | 29 |
| | Negative | 0 | 381 | 381 |
| Total | | 27 | 383 | 410 |

TABLE 7
Overall sample negative and positive statistical table

Control method

| | | | |
|---|---|---|---|
| Experiment | Positive | Negative | Total |

| | | | | |
|---|---|---|---|---|
| Kit to be assessed | Positive | 214 | 8 | 222 |
| | Negative | 3 | 185 | 188 |
| Total | | 217 | 193 | 410 |

### Example 4. Sensitivity of the Composition of the Present Invention

Experiments were carried out with samples of different concentrations to test the sensitivity of the composition of the present invention.

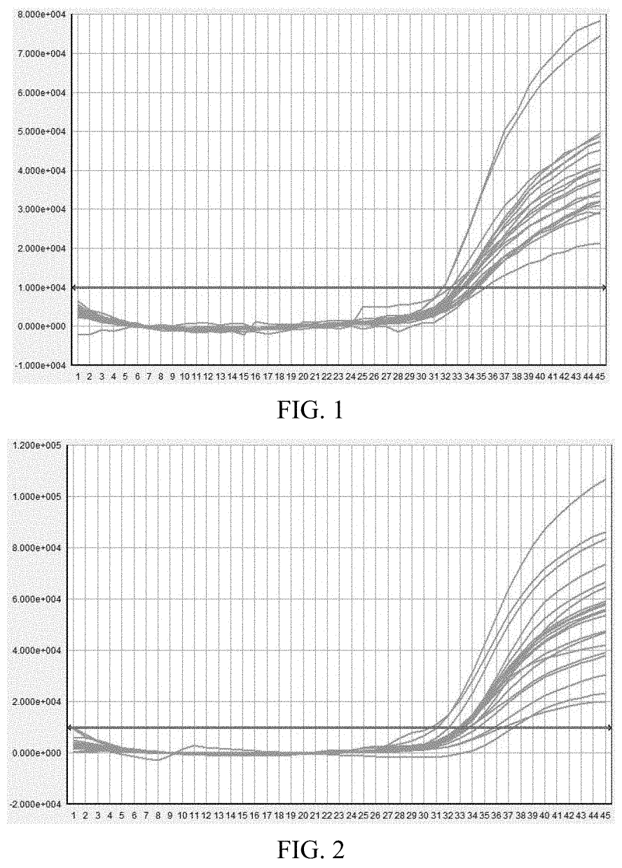

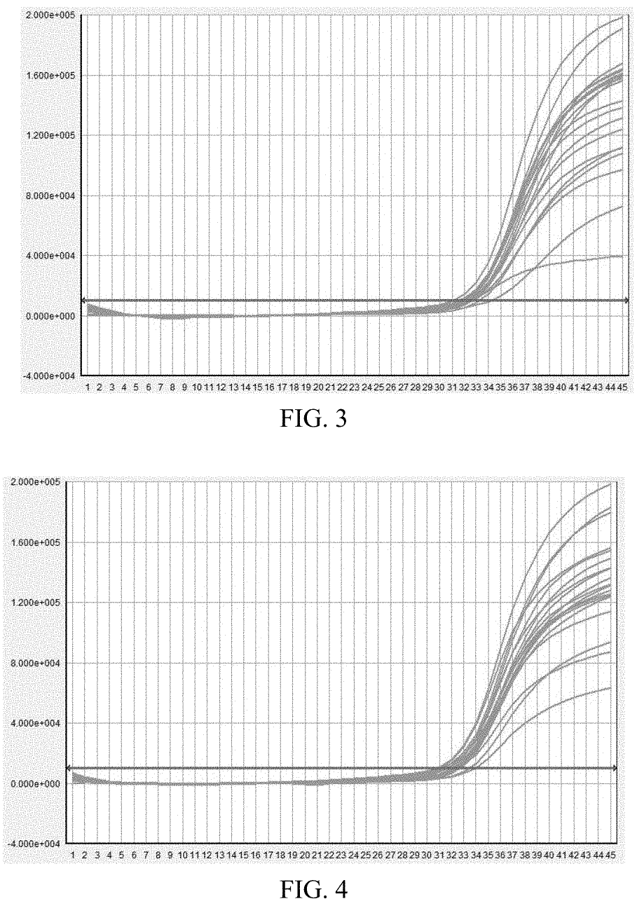

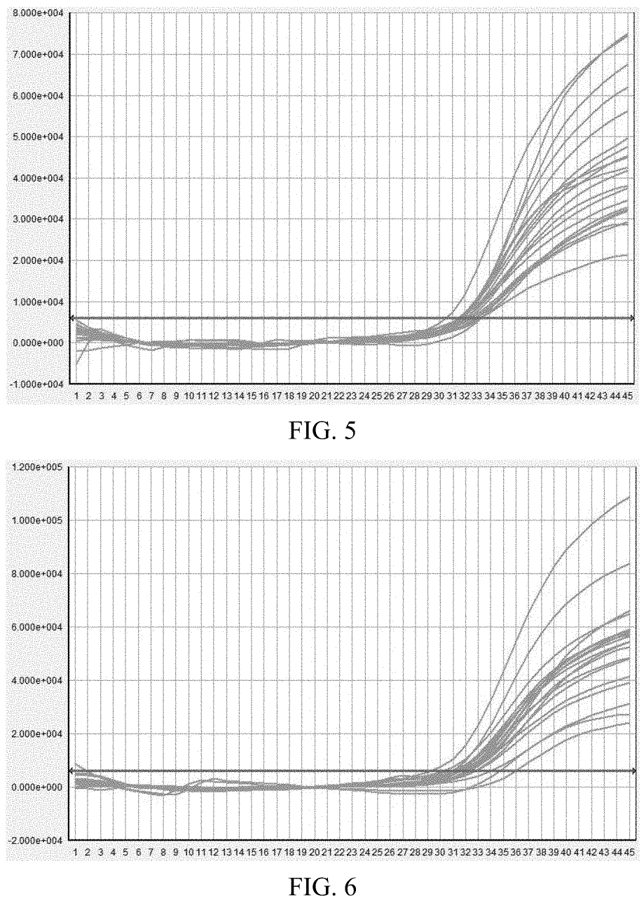

Using the composition in Table 1 of the present invention, according to the method described in Example 2, each target sensitivity positive sample was subjected to detection gradient dilution, and the sample diluted to a detection limit was used as a test sample for detection, and the detection was carried out in each channel 20 times. The composition of the present invention had a detection sensitivity of 400 copies/mL for detection of a 2019 novel coronavirus, a detection sensitivity of 6.5 TCID50/mL for detection of an influenza A virus, a detection sensitivity of 3.0 TCID50/mL for detection of an influenza B virus, a detection sensitivity of 500 copies/mL for detection of a respiratory syncytial virus, a detection sensitivity of 400 copies/mL for detection of an adenovirus, and a detection sensitivity of 400 copies/mL for detection of Mycoplasma pneumoniae. The experimental results are as shown in FIGS. 1-6.

### Example 5. Specificity of the Composition of the Present Invention

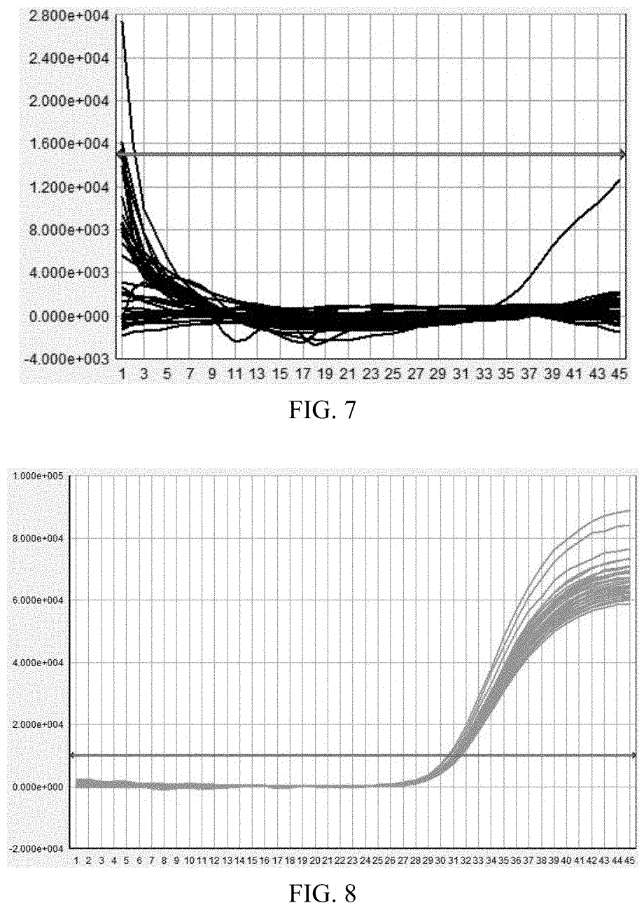

The composition of the present invention was used to detect other common respiratory tract pathogens. Experiments showed that the composition of the present invention had no cross-reaction with common respiratory tract pathogens (measles virus, mumps virus, rubella virus, Staphylococcus aureus, Escherichia coli, Pseudomonas aeruginosa, human parainfluenza virus type I, cytomegalovirus, Coxsackie virus type A, human metapneumovirus, Bacillus pertussis, Chlamydia pneumoniae, Haemophilus influenzae, Streptococcus salivarius, Streptococcus pneumoniae, Neisseria meningitidis, Mycobacterium tuberculosis, etc.). The experimental results are as shown in FIG. 7.

### Example 6. Effects of Interfering Substances on the Composition of the Present Invention

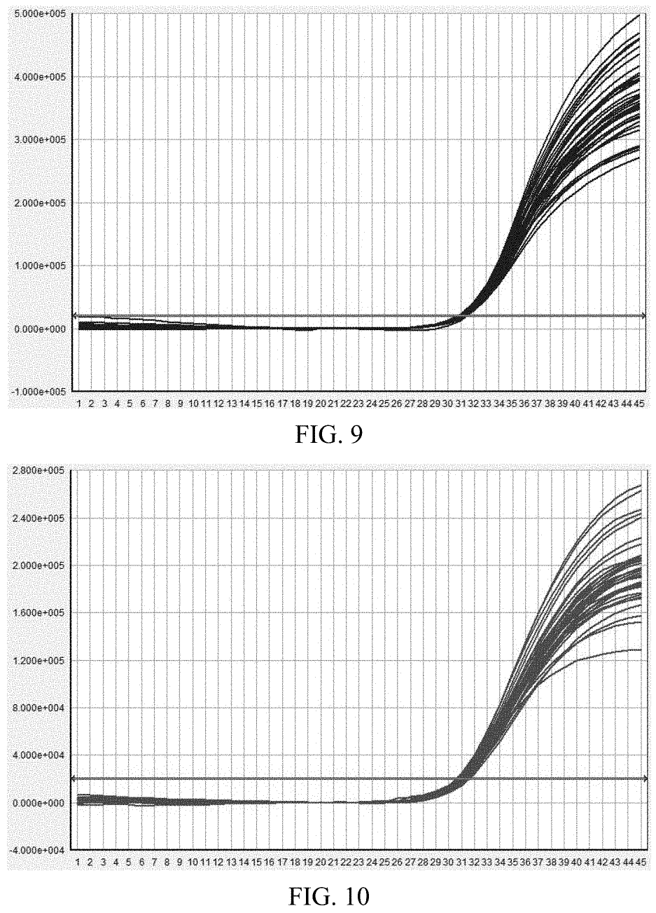

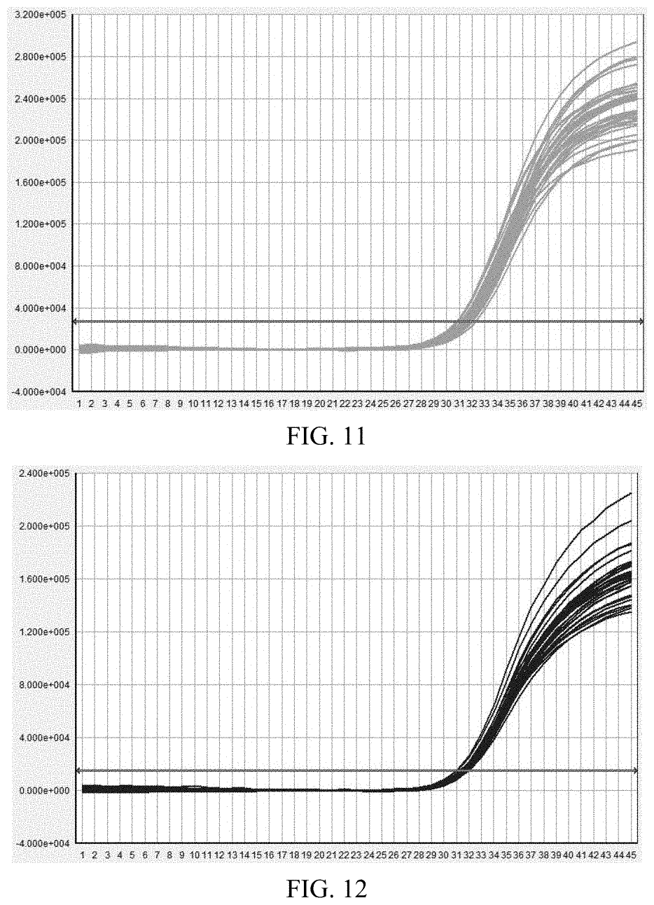

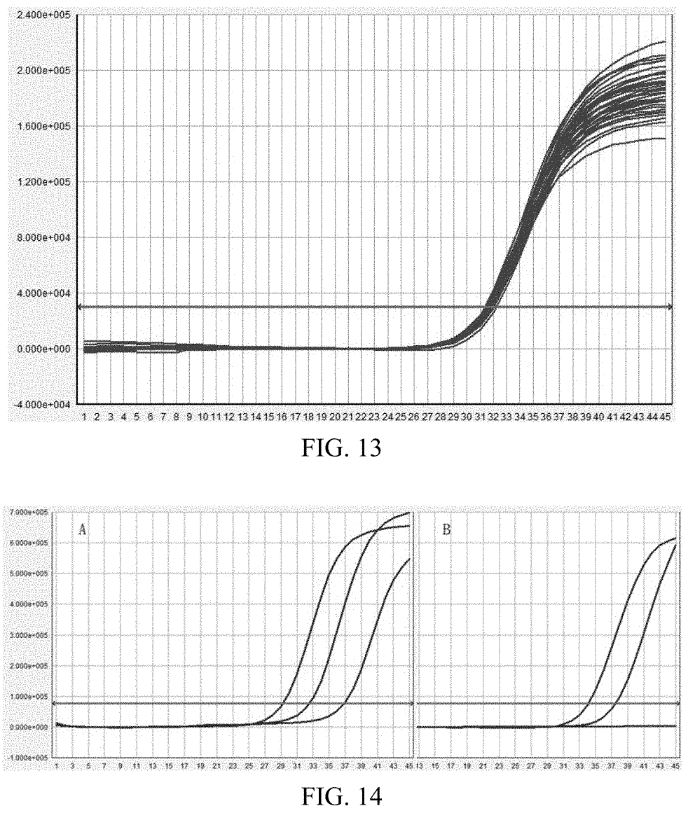

The detection with this kit was not affected by the addition of a certain proportion of a therapeutic drug such as a common cold medicine, a glucocorticoid, an antibiotic, etc. respectively into the samples. Samples containing oxymetazoline hydrochloride (100 μg/mL), dexamethasone (50 μg/mL), cefmenoxime hydrochloride (50 μg/mL), menthol (50 μg/mL), zanamivir (100 μg/mL), ribavirin (100 μg/mL), and azithromycin (100 μg/mL) were tested respectively. The results showed that these interferences had no effect on the detection results and did not affect the accuracy of the composition of the present invention. The experimental results are as shown in FIGS. 8-13.

### Example 7. Joint Detection of Multiple Pathogens, and Mutual Interference Between Primers and Probes

Due to the principle of complementary base pairing, a dimer will be formed between primers and/or probes, but this probability is low and can be ruled out at the beginning of design. However, when multiple pathogens are jointly detected, there are numerous primers and probes, and the dimer is prone to form between a primer and a primer, a probe and a probe, or a primer and a probe. In order to ensure the conservation of the design (conservation is critical to the accuracy of detection) and take mutual interference between different primers and probes into consideration, the primers and probes need to be carefully designed.

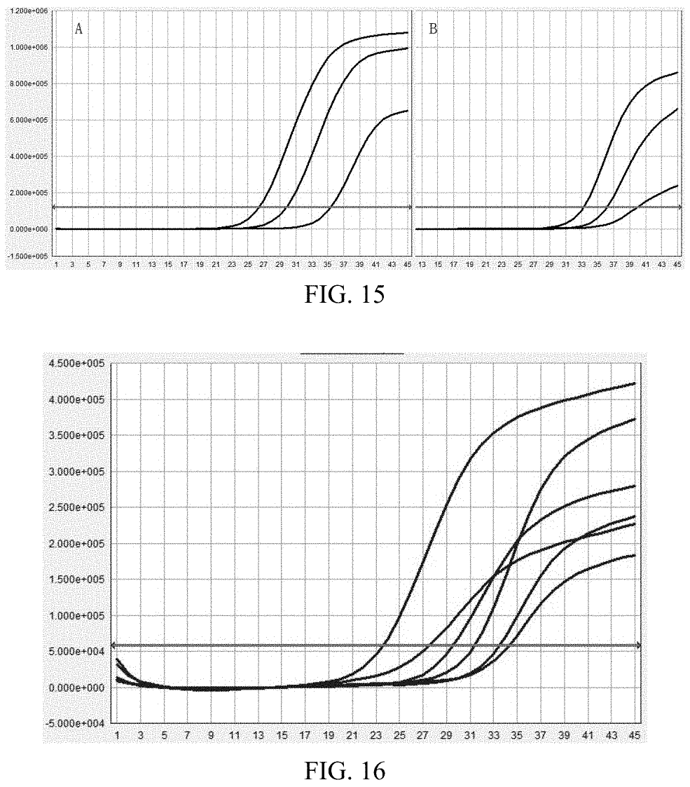

In the present invention, the inventor replaced the Mycoplasma pneumoniae in the present invention with a rhinovirus or a metapneumovirus, but through tests, it was found that the addition of the rhinovirus and the metapneumovirus could seriously affect the detection sensitivity of the composition and method of the present invention (the experimental results are shown in FIG. 14 and FIG. 15). It can be seen from the figures that after using the rhinovirus or the metapneumovirus instead, the Ct value shifted backwards significantly, which would affect the sensitivity of the composition and method of the present invention.

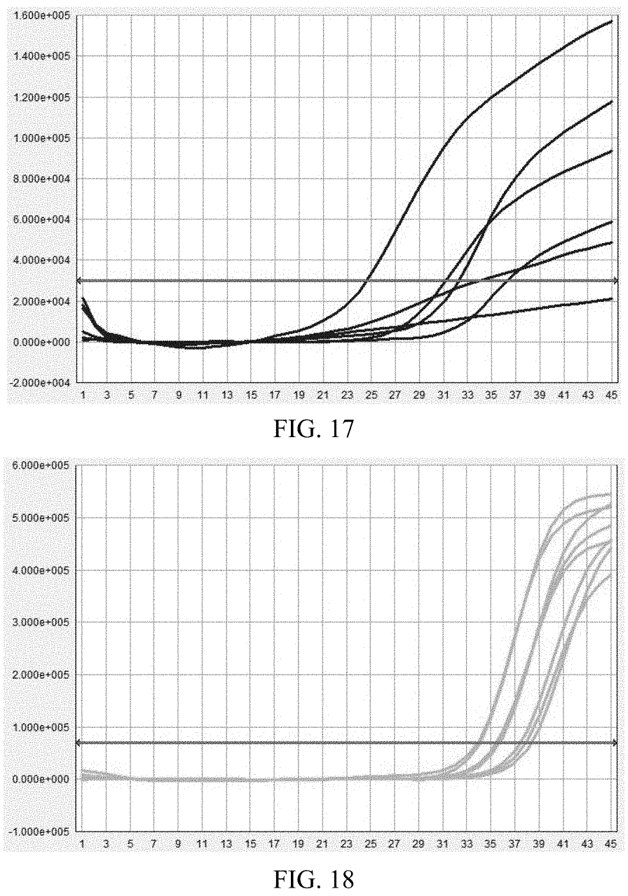

In the present invention, the inventor compared preferred primers and probe for detecting an influenza A virus in the present invention with other primers and probe for detecting an influenza A virus designed by the inventor (the primer sequences were SEQ ID NO: 23: GCGGATGCCTTTTGTTGATT and SEQ ID NO: 24: TTGCTTCAAATGAGAATGTGG; the probe sequence was SEQ ID NO: 25: CCACTCCTGGTCCTTATGGCCCAGT), and simultaneously tested six influenza A virus samples. The experimental results are as shown in FIG. 16 and FIG. 17. The results showed that the other primer and probes not preferred in the present invention seriously affected the detection results of the entire system.

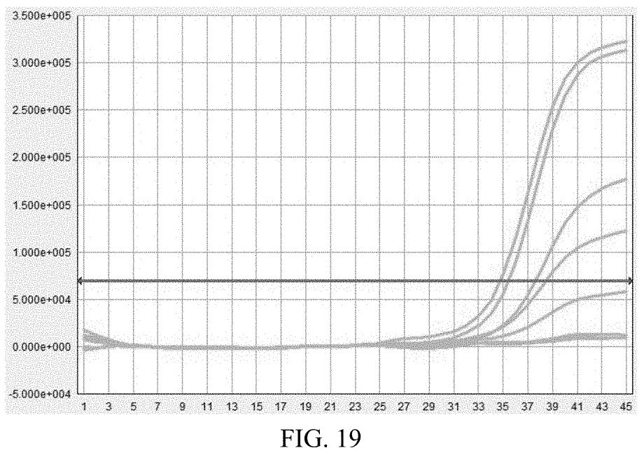

In the present invention, the inventor compared preferred primers and probe for detecting a novel coronavirus 2019-nCoV in the present invention with other primers and probe designed by the inventor for detecting a novel coronavirus 2019-nCoV (the primer sequences were SEQ ID NO: 26: CACTGATGACAATGCGTTAGCTTAC and SEQ ID NO: 27: CCAGTTCTGTATAGATAGTACCAGTTCCA; the probe sequence was SEQ ID NO: 28: CGGATAACAGTGCAAGTACAAACCTACCT). The concentration of a novel coronavirus 2019-nCoV standard was diluted into three gradients (2000 copies/mL, 500 copies/mL, and 200 copies/mL). Experimental results are as shown in FIG. 18 and FIG. 19. The results showed that the other primers and probes not preferred in the present invention seriously affected the detection results of the entire system.

Therefore, the composition of the present invention is unique.

What is claimed is:

1. A composition capable of detecting and typing pathogens that cause a respiratory infection, the composition comprising:
a first nucleic acid composition:
an influenza A virus forward primer as shown in SEQ ID NO: 1, an influenza A virus reverse primer as shown in SEQ ID NO: 2, and an influenza A virus probe as shown in SEQ ID NO: 3;
an influenza B virus forward primer as shown in SEQ ID NO: 4, an influenza B virus reverse primer as shown in SEQ ID NO: 5, and an influenza B virus probe as shown in SEQ ID NO: 6;
a respiratory syncytial virus forward primer as shown in SEQ ID NO: 7, a respiratory syncytial virus reverse primer as shown in SEQ ID NO: 8, and a respiratory syncytial virus probe as shown in SEQ ID NO: 9; and
a second nucleic acid composition:
a respiratory adenovirus forward primer as shown in SEQ ID NO: 10, a respiratory adenovirus reverse primer as shown in SEQ ID NO: 11, and a respiratory adenovirus probe as shown in SEQ ID NO: 12;
a novel coronavirus 2019-nCoV forward primer as shown in SEQ ID NO: 13, a novel coronavirus 2019-nCoV reverse primer as shown in SEQ ID NO: 14, and a novel coronavirus 2019-nCoV probe as shown in SEQ ID NO: 15;
a Mycoplasma pneumoniae forward primer as shown in SEQ ID NO: 16, a Mycoplasma pneumoniae reverse primer as shown in SEQ ID NO: 17, and a Mycoplasma pneumoniae probe as shown in SEQ ID NO: 18;
wherein fluorescent reporter groups of the first nucleic acid composition are different from each other and do not interfere with each other, and fluorescent reporter groups of the second nucleic acid composition are different from each other and do not interfere with each other.

2. The composition according to claim 1, wherein the composition further comprises an internal standard forward primer, internal standard reverse primer, and internal standard probe for monitoring.

3. The composition according to claim 2, wherein the internal standard forward primer is as shown in SEQ ID NO: 19, the internal standard reverse primer is as shown in SEQ ID NO: 20, and the internal standard probe is as shown in SEQ ID NO: 21.

4. The composition according to claim 1, wherein the fluorescent reporter groups are selected from FAM, HEX, ROX, VIC, CY5, 5-TAMRA, TET, CY3, and JOE.

5. The composition according to claim 1, wherein a fluorescent reporter group of the influenza A virus probe as shown in SEQ ID NO: 3 is FAM; a fluorescent reporter group of the influenza B virus probe as shown in SEQ ID NO: 6 is HEX; a fluorescent reporter group of the respiratory syncytial virus probe as shown in SEQ ID NO: 9 is CY5; a fluorescent reporter group of the respiratory adenovirus probe as shown in SEQ ID NO: 12 is FAM; a fluorescent reporter group of the novel coronavirus 2019-nCoV probe as shown in SEQ ID NO: 15 is HEX; and a fluorescent reporter group of the Mycoplasma pneumoniae probe as shown in SEQ ID NO: 18 is CY5.

6. The composition according to claim 1, wherein an amount of detection primer in the composition is 25 nM-500 nM; and an amount of detection probe in the composition is 20 nM-500 nM.

7. The composition according to claim 2, wherein an amount of the internal standard primer in the composition is 12.5 nM-250 nM; and an amount of the internal standard probe in the composition is 10 nM-250 nM.

8. The composition according to claim 1, wherein two nucleic acid compositions are each present in separate packages.

9. The composition according to claim 1, wherein components of the first nucleic acid composition or the second nucleic acid composition are present in a mixed form.

10. A kit for detecting and typing pathogens that cause a respiratory infection, the kit comprising the composition according to claim 1.

11. The kit according to claim 10, wherein the kit further comprises at least one of a nucleic acid release reagent, a dNTP, a reverse transcriptase, a DNA polymerase, a PCR buffer, and Mg²⁺.

12. The kit according to claim 11, wherein the amount of the detection primer in the composition is 25 nM-500 nM; and the amount of the detection probe in the composition is 20 nM-500 nM.

13. The kit according to claim 11, wherein the amount of the internal standard primer in the composition is 12.5 nM-250 nM; and the amount of the internal standard probe in the composition is 10 nM-250 nM.

14. The kit according to claim 11, wherein an amount of the dNTP is 0.2 mM-0.3 mM.

15. The kit according to claim 11, wherein a concentration of the reverse transcriptase is 5 U/μL to 15 U/μL.

16. The kit according to claim 11, wherein a concentration of the DNA polymerase is U/μL to 15 U/μL.

17. A method for detecting and typing pathogens that cause a respiratory infection, the method comprising the steps of:
1) releasing a nucleic acid of a testing sample;
2) performing, by using the composition according to claim 1, a fluorescent quantitative PCR on the nucleic acid obtained in step 1); and
3) obtaining and analyzing results.

18. The method according to claim 17, wherein the testing sample is a throat swab, sputum, a bronchoalveolar lavage fluid, and blood.

19. The method according to claim 17, wherein reaction conditions of the fluorescent quantitative PCR are as follows:
pre-denaturation and enzyme activation at a temperature of 95° C. for 1-10 minutes for 1 cycle; reverse transcription at a temperature of 60° C. for 25-35 minutes for 1 cycle; cDNA pre-denaturation at a temperature of 95° C. for 1-10 minutes for 1 cycle; denaturation at a temperature of 95° C. for 10-20 seconds; annealing at a temperature of 60° C. for 20-40 seconds for 40-50 cycles.

20. A method for diagnosing a novel coronavirus 2019-nCoV, the method comprising the steps of:
1) releasing a nucleic acid of a testing sample;
2) performing, by using the composition according to claim 1, a fluorescent quantitative PCR on the nucleic acid obtained in step 1); and
3) obtaining and analyzing results, if a typical S-shaped amplification curve is detected in a fluorescent reporter groups channel of the novel coronavirus 2019-nCoV probe shown in SEQ ID NO:15, and Ct<39, a detection result of the novel coronavirus 2019-nCoV is indicated as positive.


SEQUENCE LISTING (28 sequences)

SEQ ID NO: 1 - DNA, 20 bp, synthetic construct
  source 1..20: mol_type = other DNA; note = Influenza A virus forward primer
cagaagatttgtccttccag

SEQ ID NO: 2 - DNA, 23 bp, synthetic construct
  source 1..23: mol_type = other DNA; note = Influenza A virus reverse primer
gtcatactcctctgcattgtctc

SEQ ID NO: 3 - DNA, 27 bp, synthetic construct
  source 1..27: mol_type = other DNA; note = Influenza A virus probe
tcttcgagctctcggacgaaaaggcaa

SEQ ID NO: 4 - DNA, 19 bp, synthetic construct
  source 1..19: mol_type = other DNA; note = Influenza B virus forward primer
gccatcggatcctcaattc

SEQ ID NO: 5 - DNA, 22 bp, synthetic construct
  source 1..22: mol_type = other DNA; note = Influenza B virus reverse primer
ttcttccgtgaccagtctaatt

SEQ ID NO: 6 - DNA, 27 bp, synthetic construct
  source 1..27: mol_type = other DNA; note = Influenza B virus probe
aaggacatccaaagccaattcgagcag

SEQ ID NO: 7 - DNA, 24 bp, synthetic construct
  source 1..24: mol_type = other DNA; note = Respiratory syncytial virus forward primer
caccctgacaaaatcaacaatata

SEQ ID NO: 8 - DNA, 22 bp, synthetic construct
  source 1..22: mol_type = other DNA; note = Respiratory syncytial virus reverse primer
cataggcacccatattgttagt

SEQ ID NO: 9 - DNA, 26 bp, synthetic construct
  source 1..26: mol_type = other DNA; note = Respiratory syncytial virus probe
agacaagatggggcaaatatggaaac

SEQ ID NO: 10 - DNA, 22 bp, synthetic construct
  source 1..22: mol_type = other DNA; note = Respiratory adenovirus forward primer
gcagtggtcttacatgcacatc

SEQ ID NO: 11 - DNA, 20 bp, synthetic construct
  source 1..20: mol_type = other DNA; note = Respiratory adenovirus reverse primer
tatcctcacggtccacaggg

SEQ ID NO: 12 - DNA, 25 bp, synthetic construct
  source 1..25: mol_type = other DNA; note = Respiratory adenovirus probe
ccaggacgcctcggagtacctgagc

SEQ ID NO: 13 - DNA, 26 bp, synthetic construct
  source 1..26: mol_type = other DNA; note = Novel coronavirus 2019-nCoV forward primer
agcaatctttaatcagtgtgtaacat

SEQ ID NO: 14 - DNA, 24 bp, synthetic construct
  source 1..24: mol_type = other DNA; note = Novel coronavirus 2019-nCoV reverse primer
aaatcacatggggatagcactact

SEQ ID NO: 15 - DNA, 26 bp, synthetic construct
  source 1..26: mol_type = other DNA; note = Novel coronavirus 2019-nCoV probe
aaagagccaccacattttcaccgagg

SEQ ID NO: 16 - DNA, 20 bp, synthetic construct
  source 1..20: mol_type = other DNA; note = Mycoplasma pneumoniae forward primer
ctttggttcgcatgaatcaa

SEQ ID NO: 17 - DNA, 18 bp, synthetic construct
  source 1..18: mol_type = other DNA; note = Mycoplasma pneumoniae reverse primer
ttccctactgctgcctcc

SEQ ID NO: 18 - DNA, 28 bp, synthetic construct
  source 1..28: mol_type = other DNA; note = Mycoplasma pneumoniae probe
tcgttatttgatgagggtgcgccatatc

SEQ ID NO: 19 - DNA, 21 bp, synthetic construct
  source 1..21: mol_type = other DNA; note = Internal standard forward primer
ctcctctgacttcaacagcga

SEQ ID NO: 20 - DNA, 18 bp, synthetic construct
  source 1..18: mol_type = other DNA; note = Internal standard reverse primer
ccaccaccctgttgctgt

SEQ ID NO: 21 - DNA, 20 bp, synthetic construct
  source 1..20: mol_type = other DNA; note = Internal standard probe
ttgacgctggggctggcatt

SEQ ID NO: 22 - DNA, 132 bp, synthetic construct
  source 1..132: mol_type = other DNA; note = The GAPDH gene sequence
tctcctctgacttcaacagcgacacccactcctccacctttgacgctggggctggcattgccctcaacgaccactttgtcaagctcatttcctggtatgacaacgaatttggctacagcaacagggtggtgg

SEQ ID NO: 23 - DNA, 20 bp, synthetic construct
  source 1..20: mol_type = other DNA; note = Other influenza A virus forward primer
gcggatgccttttgttgatt

SEQ ID NO: 24 - DNA, 21 bp, synthetic construct
  source 1..21: mol_type = other DNA; note = Other influenza A virus reverse primer
ttgcttcaaatgagaatgtgg

SEQ ID NO: 25 - DNA, 25 bp, synthetic construct
  source 1..25: mol_type = other DNA; note = Other influenza A virus probe
ccactcctggtccttatggcccagt

SEQ ID NO: 26 - DNA, 25 bp, synthetic construct
  source 1..25: mol_type = other DNA; note = Other novel coronavirus 2019-nCoV forward primer
cactgatgacaatgcgttagcttac

SEQ ID NO: 27 - DNA, 29 bp, synthetic construct
  source 1..29: mol_type = other DNA; note = Other novel coronavirus 2019-nCoV reverse primer
ccagttctgtatagatagtaccagttcca

SEQ ID NO: 28 - DNA, 29 bp, synthetic construct
  source 1..29: mol_type = other DNA; note = Other novel coronavirus 2019-nCoV probe
cggataacagtgcaagtacaaacctacct